(19) United States
(12) Patent Application Publication
(10) Pub. No.: US 2006/0234269 A1
(43) Pub. Date: Oct. 19, 2006

(54) Laser Modification and Functionalization of Substrates

(76) Inventors: Matthew Asplund, Provo, UT (US); Matthew Linford, Orem, UT (US); Guilin Jiang, Seattle, WA (US)

Correspondence Address: GREENLEE WINNER AND SULLIVAN P C, 4875 PEARL EAST CIRCLE, SUITE 200, BOULDER, CO, 80301, US

(21) Appl. No.: 11/379,217

(22) Filed: Apr. 18, 2006

Related U.S. Application Data
(60) Provisional application No. 60/672,906, filed on Apr. 18, 2005.

(51) Int. Cl. C12Q 1/68; B05D 3/02

(52) U.S. Cl. 435/6; 427/2.11

(57) ABSTRACT

Assay devices comprising substrates functionalized to comprise probe species on multiple separate regions are provided. Ten thousand to a hundred thousand separate regions can be provided in a substrate of one square centimeter. The separate regions can comprise separate probe species, or in another embodiment, multiple different probe species can be present on each single functionalized region. The probe species are selected to be specific for binding to target species of interest in a sample. Methods and systems for making these devices are also provided. The devices are useful, for example for assaying molecules in a human sample that are reactive to a large number of different allergens placed on the substrate.

### CROSS-REFERENCE TO RELATED APPLICATIONS

This application claims priority to U.S. Provisional Patent Application Ser. No. 60/672,906 filed Apr. 18, 2005, which is incorporated herein by reference to the extent not inconsistent herewith.

### BACKGROUND OF THE INVENTION

Microarray technology has been evolving rapidly for more than a decade and is proving to be a valuable tool in studies requiring the use of many probe species or detection sites in order to elucidate the identification of a particular individual, organism, disease, mutation, antibody, antigen, etc. Microarrays have made significant impacts in the fields of genomics and proteomics as well as other areas of science and biotechnology. Most microarray technologies have been reliant on nylon or nitrocellulose membranes or have been coated with a specialized coating, e.g., polylysine, which prepares the surface and makes it amenable to binding of probe species such as oligonucleotides or proteins. The manufactured probe species are often added by pin spotting or other liquid handling method or they are created directly on the substrate as in the case of microarrays created using photolithography.

Bhatia, S. K. et al. (1992), “New Approach to Producing Patterned Biomolecular Assemblies,” J. Am. Chem. Soc. Discloses patterning a thin film using photolithography with UV light exposure through a mask. Calvert, J. M., et al. (1992), “Deep UV photochemistry and patterning of self-assembled monolayer films,” Thin Solid Films 210/211:359-363 also discloses using a mask to pattern an organosilane self-assembled monolayer using UV irradiation. U.S. Pat. No. 5,648,201 issued Jul. 15, 1997 to Dulcey et al. for “Efficient Chemistry for Selective Modification and Metallization of Substrates, also discloses the use of a mask for patterning with actinic radiation. U.S. Pat. No. 5,688,642 issued Nov. 18, 1997 to Chrisey et al. also discloses patterning a coated substrate by irradiation through a mask. U.S. Pat. No. 6,436,615 issued Aug. 20, 2002 to Brandow et al. for “Methods and Materials for Selective Modification of Photopatterned Polymer Films discloses irradiation with excimer lasers through a mask for patterning.

Patterning of substrates using scanning lasers is disclosed in U.S. Pat. No. 5,057,184 issued Oct. 15, 1991 to Gupta et al. for “Laser Etching of Materials in Liquids. This patent discloses a method of etching using sonic cavitation. U.S. Patent Publication No. 2003/0080089 published May 1, 2003 of Song et al. for “Method of Patterning a Substrate,” discloses patterning a substrate with a laser scanning along a predetermined path. Balgar, T. et al. (2006) “Laser-assisted decomposition of alkylsiloxane monolayers at ambient conditions: rapid patterning below the diffraction limit,” Appl. Phys. A82:689-695 discloses patterning created by a scanning laser via ablation. U.S. Patent Publication No. 2004/0058059 published Mar. 25, 2004 of Linford et al. for “Functionalized Patterned Surfaces” teaches a method of functionalizing the surface of a material that has been patterned by scribing with an instrument to form a surface capable of reacting with a reactive species.

Srinivasan, R. and Braren, B. (1989), “Ultraviolet Laser Ablation of Organic Polymers,” Chem. Rev. 89:1303-1316 discusses the phenomenon of laser ablation of polymeric substrates, but does not describe methods for patterning surfaces. Bityurin, N. (2005), “8 Studies on laser ablation of polymers,” in Annu. Rep. Prog. Chem., Sect. C 101:216-247 discusses ablation patterns and random, non-useful surface features such as ripples produced by laser ablation of polymeric substrates.

Some of the basic challenges associated with microarrays, in addition to coating the substrate or slide with specific probes, pertain to specificity, sensitivity, cost, and ease of use and manufacture.

With the development of the lithographic techniques necessary to produce micro-optics have come new developments in their use. Companies including Suss MicroOptics, Omron, MEMS Optical and Epigem all produce arrays of lenses, with typical lens sizes of 100 microns, and devices with 1000-10000 lenses. There are currently four primary uses for these arrays. The first is as a beam homogenizer. This system is used primarily in microscope illumination to get rid of spatial variations in light intensity, and to get rid of interference patterns in laser illumination. The second major use for the arrays is for fiber coupling. Fiber optics have cores whose sizes are similar to the lens array element sizes, and so light can be broken up into “beamlets” or small sections of the input beam, which can be coupled into arrays of individual fibers and then used for imaging. The third use is for direct imaging. Recent work has shown the direct coupling of these arrays onto imaging chips, with one micro-lens per pixel. The final use is the connection of lens arrays with micromachines. Many micro-chemical systems depend on fluorescence emission to provide a signal from a sample volume, which is hundreds of microns in diameter. By bonding a microlens array directly onto a micromachined chemical chip, the alignment of fluorescence collection optics is guaranteed. U.S. Pat. No. 6,822,799 issued Nov. 23, 2004 to Kitamura et al. for “Exposing Apparatus and Exposing Method for Microlens Array teaches a method for making a microlens array. No use of microlens arrays for focusing and transmitting light onto a substrate for the purpose of functionalizing the substrate is known to the inventors hereof.

There is a need in the art for functionalized substrates having greater sensitivity and specificity that cost less per unit than currently-available substrates.

All publications referred to herein are incorporated by reference herein to the extent not inconsistent herewith for the purpose of providing description and enablement of aspects of the methods and systems of the present invention.

### SUMMARY OF THE INVENTION

The present invention provides functionalized substrates (substrates are also referred to herein as chips, microchips, and slides) for use as assay devices for the detection of target species. The substrates of this invention are designed to have greater sensitivity and specificity at a much lower cost per slide than is currently available. Because only a specific region of the substrate is being functionalized, rather than the entire slide or chip, the signal to noise ratio is significantly better than with most existing technologies. In addition, because a greater number of sites are being functionalized within a given region of the substrate, the binding of probe species is much more efficient and effective in comparison to existing platforms of a similar design. Finally, the slides are designed to be produced at very low costs per slide and used at a lower cost per determination. In the case of genotype analysis, DNA probes are bound to the slide within a specified region and with great affinity to the functionalized site, and many different probe species can be bound to a single spot within an array having several thousand spots per slide/chip.

The invention features new, straightforward methods for functionalizing multiple regions of a substrate. For example, the present invention features a new technique for patterning and functionalizing multiple spatially-separated regions of a substrate. This functionalization requires only a single laser pulse, which can simultaneously functionalize multiple regions. The laser powers employed are easily obtained with commercially-available, low-cost lasers.

### BRIEF DESCRIPTION OF THE DRAWINGS

FIG. 1 depicts a method for functionalizing a substrate and preparing the substrate for attaching a probe species.

FIG. 2 depicts ToF-SIMS negative ion images of functionalized spots on Si made under (a) 1-hexene (b) 1-decene (c) 1-tetradecene (d) octane, and (e) Ge under 1-iodooctane. A total ion image and the image of the first principal component from a principal components analysis using the instrument software is shown for the functionalized spot on Ge.

FIG. 3 depicts negative-ion AXSIA spectral images, a composite image of AXSIA components 1-3 (C1-C3), and single ion images of ToF-SIMS of silicon surfaces modified with a laser using 1-decene (top images). Spectra of AXSIA components of functionalized spots on silicon modified with 1-hexadecene (bottom spectra).

FIG. 4 depicts XPS survey spectra of a silicon surface that had been wet with 1-hexadecene. Top: blank region that was not exposed to a pulse of laser light. Bottom: functionalized spot.

FIG. 5 depicts XPS scans of functionalized spots on Si made under 1-hexadecene and control (unfunctionalized) regions near the functionalized spots. a) C 1s functionalized spot, b) C 1s control, c) Si 2p functionalized spot and d) Si 2p control.

FIG. 6 depicts:Top: AFM height image of a small functionalized spot. Bottom: Trace through the middle of the spot.

### DETAILED DESCRIPTION

According to the present invention, light is directed through a lens array, preferably a microlens array, onto multiple regions of a substrate, which light causes a change of functionality of the multiple regions resulting in multiple primarily-functionalized regions that are spatially separated. When a pulse of laser light is used functionalization of the multiple spatially separated regions can be achieved within about 0.1 picosecond to about 100 microseconds, or in embodiments of this invention, within about 1 nanosecond to about 1 microsecond, or in other embodiments, about 1 nanosecond to about 10 nanoseconds, and in other embodiments of this invention from about 4 to about 7 nanoseconds.

The primarily-functionalized regions can then be further functionalized by (1) exposing the primarily-functionalized regions to a first chemical environment which causes the primarily-functionalized regions to undergo a secondary change in functionality resulting in multiple secondarily-functionalized regions; (2) removing the first chemical environment leaving exposed the multiple secondarily-functionalized regions; (3) optionally, repeating steps (1) and (2) with second and subsequent chemical environments until desired multiple terminally functionalized regions are achieved. One or more probe species may then be attached to the multiple spatially-separated terminally functionalized regions. “Terminally-functionalized” is used herein to refer to regions having reactive groups that will bind desired probe species.

The chemical reactions that are necessary for preparing an assay device are simplified by the present invention. For example, the substrate can be exposed to the first chemical environment simultaneous with directing the light through the microlens array. When the first pulse of light creates multiple primarily-functionalized regions per square centimeter, the first chemical environment immediately reacts with multiple primarily-functionalized regions of the substrate resulting in multiple secondarily-functionalized regions that are spatially-separated. In this way, as many as about 10,000 to about 100,000 functionalnized regions per square centimeter are created with a single pulse of light, or in some embodiments about 10,000 to about 50,000 functionalized regions per square centimeter are created by the single pulse of light.

Although various first chemical environments can be used in the present invention, the first chemical environment can be selected such that one or more probe species can be directly attached to the secondarily-functionalized regions without a complex, multiple-step chemical reaction process.

In another aspect of the present invention, the substrate can comprise a hydrophobic background layer and an underlying layer that is hydrophilic or can be made hydrophilic by action of focused light thereon. A hydrophobic background layer allows localization of liquid chemical environments to spatially-separated functionalized regions, because a aqueous liquid chemical environment will concentrate on areas of the substrate where the hydrophobic background layer has been removed, leaving the remainder of the substrate free of the liquid. In other emodiments, the substrate comprises a hydrophilic background layer and an underlying layer that is hydrophobic or can be made hydrophobic by action of focused light thereon. This allows localization of liquid chemical environments to spatially-separated functionalnized regions because a non-polar liquid chemical environment will concentrate on areas of the substrate where the hydrophilic background layer has been removed and the hydrophobic underlying layer has been exposed. In one embodiment, the light changes the functionality of multiple regions of the substrate by heating the underlying layer, which removes portions of the background layer leaving a primarily-functionalized region. The primarily-functionalized region can be functionalized by the pulse of light by virtue of exposing its underlying layer layer, or can be functionalized by the creation of reactive groups such as silicon or oxygen radicals on the substrate.

The pulse of laser light can be of such a duration and energy that the substrate is functionalized by removing the background layer without melting the underlying substrate, resulting in a primarily-functionalized region. In other embodiments, the pulse of laser light can partially or completely melt the underlying layer of the exposed region without removing a measurable amount of material from the underlying layer. In another embodiment, ablation of the substrate may take place. The primarily-functionalized region is secondarily-functionalized when functional groups on the primarily-functionalized region react with a chemical environment to produce further functional groups attached to the region.

Substrates useful in this invention include those comprising materials selected from the group consisting of silicon, glass, diamond, including hydrogen-terminated diamond and diamond with an oxidized surface, as well as diamond coated with a monolayer such as an alkyl monolayer, e.g., composed of alkyl chains having about 1 to about 22 carbon atoms, straight or branched, and optionally comprising reactive groups as listed below with respect to reactive groups supplied by chemical environments used in this invention. Substrates can also comprise polycarbonate, fused silica, germanium, silane monolayers, alkene monolayers, thiol monolayers, Teflon™, metals, polyelectrolyte films, silicon nitride, silicon carbide, polydimethylsiloxane, polymethylmethacrylate, and other materials known to the art for use in manufacturing assay devices for use in detecting the presence of target species. The substrate can be in the form of a wafer, a thin film, or a monolayer, a chip or slide. A “thin film” as used herein, can be made of any material known to the art to be useful for the purposes of this invention, including polymeric and oligomeric materials, having a thickness as known to the art, typically between about 0.5 nm and about 100 micrometers. A “monolayer,” as is known to the art, is a layer having an average thickness of about one molecule.

One aspect of the present invention is the ability to simultaneously produce high-density multiple functionalized regions on a substrate in a period of time as short as about 0.1 picsecond to about 100 microseconds. Another aspect of the present invention is the simplification of the chemical reaction process traditionally needed to attach probe species to multiple functionalized regions. In previously-known methods, it is common to coat the whole surface of a substrate with reactive molecules, then place spots comprising reactants where desired, and rendering the remainder of the surface non-reactive. This invention places all reactants needed for the desired functionalization only on separated regions.

In the present process, DNA molecules to be used as probe species can be furnished with an amine group at one end and reacted with amine-reactive molecules on separated regions of the substrate, leaving the other end of the DNA molecule to react with further species such as target molecules in a sample. Amino-modified oligonucleotides have been routinely employed in solid support and label (or functionality) attachment chemistries. Such molecules can be prepared by methods known to the art, and are commercially available, e.g., from Fidelity Systems, Gaithersburg, Md. The 5′-terminus of the oligonucleotide is normally the target end for modification because of the ease of incorporation as the last step in automated synthesis. For some applications factors, such as sterics, electrostatic repulsion, binding kinetics and hybridization efficiency, require a longer distance between the oligo and the point of attachment. 5′-amino linkers are commercially available in a variety of tethering arms, based on their length, charge density, hydrophobicity, flexibility, and multiplicity of amino groups on the tether. Customized tethers and libraries of oligonucleotides with differently tethered functionalities can be created in accordance with the needs of the system.

Another aspect of the present invention includes optimizing the density of reactive molecules on the separated functionalized regions of the substrate surface. Optimal density means as much density of reactive species as is necessary to produce a good signal for detection, but not so much that bonding of the probe species with target molecules is interfered with, or so much that that fluorescence quenching occurs (as is known to the art, fluorescence dyes placed to closely together can quench each other).

Another aspect of the present invention includes increasing the detectability of fluorescing target species by depositing a fluorescence-quenching background layer on the assay device. Fluorescence-quenching substances are known to the art for various fluorescent molecules. The fluorescence-quenching materials can be added to the material used to render the background layer hydrophobic or hydrophilic, e.g., polyethylenamine with hydrocarbon chains grafted to the amine groups.

Another aspect of the present invention includes maximizing the exposed surface area of the functionalized regions without increasing the overall size of the functionalized regions by increasing the density of reactive groups on a functionalized region by making it rough at the nanoscale. Yet another aspect of the present invention is simultaneously functionalizing multiple exposed regions of a substrate with a pulse of laser light, which pulse of laser light melts and functionalizes the exposed regions without causing loss of material from the substrate.

In a method of this invention, light, such as a pulse of laser light, is directed through a lens array onto a substrate causing a change in functionality of portions of the substrate. The microlens array divides and focuses the light onto multiple spatially-separated regions of a substrate. The lens array can be a microlens arrays chosen from any commercially available microlens arrays, or a specially-designed microlens array. Microlens arrays are generally 1 mm to 10 mm in width and length with individual lenses ranging in size from about 20 micrometers to about 500 micrometers, but can be customized to any necessary size with any number of lenses. Commercially available microlens arrays can have as many as 50,000 lenses per square centimeter (also known as elements) in a single array and can be customized to have even more lenses, e.g., up to about 100,000 lenses per square centimeter in a single array. Microlens arrays are generally refractive, although reflective microlens arrays are also contemplated. Microlens arrays can be chosen with various shapes of lenses depending on the desired patterns of functionalized regions as is known to the art. For example, circular microlenses create circular functionalized regions while cylindrical microlenses create elongated functionalized regions that can be in the shape of straight lines.

According to the present invention the microlens array divides and focuses a beam of a light onto multiple spatially-separated regions of a substrate, which causes a change in functionality where the multiple divided beams of light come into contact with the surface of the substrate such that there is no overlap between the separate functionalized regions on the surface. Varying the distance between the microlens array and the substrate affects the size of the functionalized regions. For example, the present invention was performed on a silicon wafer using a 2500-element microlens array from Suss MicroOptics (Neuchatel, Switzerland). The spacing between the centers of adjacent lenses was 100 micrometers. The radius of curvature of each microlens was 120 micrometers. The lenses were rectangularly packed. When a pulse of light from a YAG laser at 532 nm was directed onto a silicon wafer using the above-described microlens array, primarily-functionalized regions with a diameter of approximately 10-20 micrometers were achieved when the laser was focused at the surface of the substrate. Primarily-functionalized regions with a diameter as small as 0.5 micrometers up to as large as 100 micrometers are possible with this technique. In embodiments of this invention the diameter of functionalized regions is between about 5 and about 20 micrometers. Diameters of functionalized regions can be as small as about 0.5 micrometers.

The light can be generated from any suitable light source known to one skilled in the art, and can be produce ultraviolet, infrared, or visible light. Preferably, the light is in the form of a pulse of visible laser light. To cause a change in functionality, the pulse of laser light should have a duration between about 0.1 picosecond to about 100 microseconds, or in other embodiments about 1 nanosecond up to about 1 microsecond, and in still other embodiments, about 1 nanosecond up to about 10 nanoseconds, and a power density between about 10⁸ and about 10¹¹ W/cm², or in some embodiments, between about 10⁹ and about 10¹⁰ W/cm².

The term “change in functionality” of a region means making the region reactive to a chemical species it did not previously react with, or increasing its reactivity with a chemical species, making the region non-reactive with a chemical species with which it previously reacted, or less reactive to a chemical species. The term “functionalized” refers to a region that has undergone a change in functionality as defined above. The term “functional group” refers to an atom or group of atoms that have the ability to react with another chemical species. Changing the functionality of a region of a silicon substrate that is oxide-terminated at the surface in its native state can include modifying the substrate in such a way so as to expose highly-reactive silicon atoms by causing degradation, oxidation, or melting of the surface so that the silicon is exposed. Similarly, with many other substrates, stripping the native material bare makes the surface more reactive.

A primarily-functionalized region refers to a region of a substrate that is initially functionalized by directing light onto that region of the substrate. Secondarily-functionalized regions, tertiarily-functionalized regions, quaternarily-functionalized regions, and subsequently-functionalized regions refer to regions whose functionalities have been successively changed by exposure to chemical environments that react with the previously-exposed functional groups to produce different functional groups.

The primarily functionalized regions can be formed by ablation, melting, or photocleavage. The following describes energies sufficient to change the functionality of multiple regions resulting in primarily-functionalized regions.

Ablation occurs when the portion of the substrate exposed to laser light heats, expands, and is ejected from the substrate. The ablation of silicon, for example, is a function of the energy deposited at the surface in a unit of time. If a typical YAG laser that has a full width at half-maximum (FWHM) of 4 ns and an energy of 50 mJ is directed through a 2500-element microlens array, with 100-micrometer spacing between adjacent lenses, and with individual lenses having a 120-micron radius of curvature, then the diameter of the divided beam at each of the exposed regions of the silicon surface is 10 micrometers with a FWHM of 4 ns and an energy of 20 mJ/cm², the power density is 10¹¹ W/cm². When this pulse of this laser light strikes the surface of a silicon substrate, a loud audible report will be heard, and a portion of the substrate is pressure ejected from the surface when it heats and expands. If the laser strikes the surface in an air environment, a plume will be ejected from the surface and will extend out from the surface a distance of a few millimeters. As is known to the art, the power density required for ablation of other substrates will be different depending on the substrate. Depending on the wavelength of the light used and the substrate material, a hole having a radius of about 100 nm to about 500 nm, or several micrometers will be ablatively removed from the exposed region. To avoid ablation power densities about an order of magnitude less than those at which ablation occurs should be used.

Cleavage, also called photocleavage, occurs when light such as ultraviolet (UV) light is directed onto a surface, which UV light causes excitation of electrons at the surface, resulting in decomposition of chemical bonds at the surface. Photocleavage procedures are typically performed on a monolayer using single photons of high enough energy to break chemical bonds. The necessary power density to break the chemical bonds at the surface can be ascertained by means known to the art based on the bond strength of the bonds to be broken.

Melting occurs when the energy and duration of the light is chosen such that the surface heats and liquefies. The melting of silicon, for example, can occur when a pulse of 532 nm light from a typical YAG laser that has a FWHM of 4 ns and an energy of 2 to 4 mJ is directed through a 2500-element microlens array, with 100-micrometer spacing between adjacent lenses, and with individual lenses having a 120-micrometer radius of curvature is directed onto a silicon wafer. The diameter of the divided beam at each of the exposed regions of the silicon wafer surface is about 5 micrometers with a FWHM of 4 ns and an energy of 0.8 mJ and the power density is 10⁹ W/cm², which is sufficient to melt the silicon without causing measurable loss of material through ablation.

Melting helps optimize coverage of the functionalized region by functional groups by creating primarily-functionalized regions that remain substantially at the surface of the substrate (a slight ripple effect is observed) instead of in holes dug in the surface by ablating. Surface functionalization with melting occurs almost instantaneously instead of requiring the longer exposure times to light that are need for photocleavage, e.g., from a few minutes up to a few hours, where 30 minutes is typical.

Power densities at the surface of a substrate sufficient to change the functionality of the surface by melting differ from substrate to substrate, and can be calculated by means known to the art from material properties, including absorbance rates for photons, temperature gradients, and melting temperatures. Also such power densities can be determined pragmatically without undue experimentation by one skilled in the art.

According to various embodiments of the present invention, the primarily-functionalized regions can be exposed to first and subsequent chemical environments depending on the desired terminally-functionalized regions. (The “desired” terminally-functionalized regions have reactive groups capable of reacting with desired probe species to cause attachment of the probe species to the region. The “desired” probe species are selected to be probe species that react with the targets in a sample that it is desired to detect. The first chemical environment causes the regions that have been primarily functionalized by the light to undergo a change in functionality resulting in a secondarily-functionalized region. In one embodiment, one or more probe species may be directly attached to the different spatially-separated secondarily-functionalized regions. In such a case, the secondarily-functionalized regions would comprise the desired terminally-functionalized regions.

In yet another embodiment, a second chemical environment would be exposed to some or all of the secondarily-functionalized regions causing the secondarily-functionalized regions to undergo yet another change in functionality resulting in a tertiarily-functionalized regions. This process can be repeated, exposing each successive functionalized region with a chemical environment that reacts with the functionalized region to produce new functional groups on the region until regions having the desired functionality are achieved. The final functionalized region, that is capable of reacting to attach the desired probe species, is referred to as the terminally-functionalized region. A probe species can then be attached to the terminally-functionalized region.

After attachment of the probe species, the substrate, functionalized with the probe species, can be contacted with a sample, such as a biological fluid, a gas, or a solid containing a target molecule that binds to the probe, whereby the presence of bound target molecule can be detected by means known to the art.

Typically, the desired terminally-functionalized regions comprise chemically-reactive moieties selected from the group consisting of at least one of amine groups, alcohol groups, epoxide groups, N-hydroxysuccinimide (NHS) ester groups, acid chloride groups, isothiocyanate groups, isocyanate groups, carboxyl groups, vinyl sulfone groups, fluorine-functionalized aromatic rings, aldehyde groups, alkyl halide groups, sulfonyl chloride groups, benzyl halide groups, aromatic rings, carbon-carbon double bonds, carbon-carbon triple bonds, methyl esters, carbodiimides, and acid anhydride groups that are capable of reacting with probe molecules known to the art, such as nucleic acids such as oligonucleotides including DNA and RNA, proteins, polypeptides, polyamides, protein-nucleic acid molecules, oligosaccharides, and reactive derivatives of these species that have been modified to contain groups reactive with the foregoing functional groups or with marker molecules known to the art.

The first chemical environment, and any subsequent environments, can be removed using methods known in the art. For example to remove some liquid chemical environments, the substrate can simply be washed, for example, using solvents for the materials in the liquid chemical environment, such as organic solvents, water, aqueous detergent solutions and the like. Surfaces can then be rinsed with deionized water. A gas chemical environment can be vacuumed or blown off, and a solid chemical environment can be melted or washed off with a solvent.

When the substrate is exposed to the first chemical environment and to the laser light simultaneously, the laser light causes a change in functionality of the multiple regions of the substrate resulting in primarily-functionalized regions. The first chemical environment immediately reacts with the primarily-functionalized regions resulting in secondarily-functionalized regions. In one variation of this invention, the first chemical environment can be an environment that under normal conditions has little or no reactivity but that can become reactive because of the energy from the pulse of light, so that it will react with the primarily-functionalized regions causing a change in functionality. For example, methane gas is not generally reactive at room temperature, but becomes reactive at the high temperatures generated by the light pulse.

The first chemical environment may contain a single or multiple chemical species. For example, ambient air may be selected as the first chemical environment such that the light passes through the lens array, then through the ambient air and then onto the substrate. Another variation includes having the light pass through a chemical environment that comprises a gaseous species, such as ambient air, and a liquid species. The liquid can be water or a solution comprising compounds having any desired reactive group. For example, the liquid can comprise water or alcohol or other liquid, and can supply functional groups selected from the group consisting of hydroxy groups, amines, alkyl halides, alkynes, carbon disulfides, epoxides, carboxylic acids, compounds having at least one aromatic ring, alkenes, NHS esters, acid chlorides, acid anhydrides, methyl esters, isocyanates, isothiocyanates, vinyl sulfones, fluorine-functionalized aromatic rings, aldehydes, carbodiimides, benzyl halides, carbon disulfide, epoxides, carboxylic acids, thiols, halides, aldehydes, ketones, amides, carboxylic acid esters, acrylates, methacrylates, vinyl ethers, acrylamides, azides, nitrites, dienes, trienes, phosphines, isocyanates, isothiocyanates, silanols, oximes, diazo, epoxides, nitro groups, sulfate groups, sulfonate groups, phosphate groups, phosphonate groups, anhydride groups, guanadino groups, phenolic groups, imines, diols, triols, hydrazones, hydrazines, disulfide groups, sulfide groups, sulfone groups, sulfoxide groups, peroxide groups, urea groups, thiourea groups, carbamate groups, diazonium groups, azo groups, DNA, RNA, protein, carbohydrates, lipids, and styrenics.

In one variation of this invention, only a liquid species is selected as the chemical environment such that the liquid species is deposited on the surface of the substrate and the lens array is then brought into contact with the liquid species. The light passes through the lens array and then passes directly through the first chemical environment and onto the substrate. This variation has the advantage that there is only a single index of refraction between the lens array and the substrate. Whereas having multiple phases in the first chemical environment results in having multiple indices of refraction and makes it more difficult to focus the light on the desired regions of the substrate.

Suitable gaseous chemical environments can be selected from, but are not limited to, the group consisting of ambient air, nitrogen, oxygen, argon, helium, ethylene, acetylene, butene, methane, and butane. Inert gases such as argon and helium are useful as chemical environments when it is desired to prevent the surface of the substrate from reacting with other materials present immediately after the pulse of laser light, for example, when it is desired to allow the surface to cool before introducing a further material with which reactive species on the surface will react.

Solid chemical environments can include compounds selected from the group consisting of compounds supplying reactive groups capable of binding further compounds having desired functionalities, and can be selected from the group consisting of polystyrene, polymethylmethacrylate, polytetrafluoroethylene, other polymers, and alkyl monolayers.

Where they are compatible, mixtures and solutions of the above species may also be used.

A wide variety of first chemical environments are known to the art as capable of functionalizing silicon. These include classes of compounds that are known to react under high or ultra-high vacuum conditions with clean, unpassivated silicon, such as alkenes, alkynes, alkyl halides, and alcohols. Unsaturated monomers also react with the exposed surface of silicon under high or ultra-high vacuum. (High and ultra-high vacuum conditions are known and defined in the art.) Other functional groups that are used in previously-known methods under high and ultra-high vacuum conditions include, but are not limited to, silanes (especially those that are hydrolyzed to contain the —OH group), amines, thiols, amine oxides, oximes, ketones, epoxides (oxiranes), aldehydes, carboxylic acids, esters, amides, lactones, lactams, nitrites, ethers, thioethers, disulfides, diacylperoxides, dialkylperoxides, and alky- or arylperoxides. The high and ultra-high vacuum conditions allow a bare substrate surface to stay clean and reactive for whatever period of time is required for the reaction to take place. This invention allows such compounds to react with silicon, germanium, diamond and other substrates without the necessity for vacuum conditions because the pulse of light on the silicon substrate creates regions of freshly-exposed native substrate material that immediately react with the compounds under the energy of the light. Thus there is no need for vacuum conditions to maintain a surface in a clean condition for long periods of time.

Vacuum conditions can, however, be used when necessary to optimize reactions performed in the processes of this invention. Vacuum chambers, pumps and related equipment required for performing reactions under vacuum conditions are known to the art.

One class of compounds that can be used to functionalize separated regions of substrate surfaces such as silicon, diamond, germanium, silicon carbide, and silicon nitride is alkenes. Any molecule with a double bond can be used for this purpose, including those with terminal double bonds, e.g., alpha-olefins, H₂C═CH(CH₂)ₙCH₃, as well as those with double bonds in other positions in the molecule. Useful alkenes include those of the general formula: H₂C═CH(CH₂)ₙX, where X is —NH₂, —COOH, —COOR (where R is an alkyl alkene, alky, or ring-containing compound having 1-50 carbon atoms, or in other embodiments 1-22 carbon atoms, in a straight or branched chain, and optionally comprising functional groups as set forth herein as useful reactive groups for functionalizing substrates), —CON H₂, —OH, —NR₃⁺, -epoxy, -glycidyl, —C₆H₆H, —C₆H₄COOH, —C₆H₄OH, —C₆H₄NH₂, -protein, -biotin, -DNA, —RNA, -polyethylene glycol (PEG), a living cell, or other moieties known to the art. Alternatively, alpha, omega.-functionalized alkenes having the formula H₂C═CH(CH₂)ₙCH═CH₂ can be used.

Another class of chemical environments that can be used to functionalize separated regions of substrate surfaces such as silicon, diamond, germanium, silicon carbide, and silicon nitride comprises alkyne compounds. The triple bond can be anywhere in the molecule, including the terminal position of an alkyl chain: HC≡C(CH₂)ₙCH₃. Alkynes of the general formula HC≡C(CH₂)ₙX, where X is —NH₂, —COOH, —COOR (where R is an alkyl, alkene, alky, or ring-containing compound having 1-50 carbon atoms, or in other embodiments 1-22 carbon atoms, in a straight or branched chain, and optionally comprising functional groups as set forth herein as useful reactive groups for functionalizing substrates), —CONH₂, —OH, —NR₃⁺, -epoxy, -glycidyl, —C₆H₅, —C₆H₄COOH, —C₆H₄OH, —C₆H₄NH₂, -protein, -biotin, -DNA, -RNA, -PEG, a living cell, or another functional group known to the art can also be used. One can also employ alpha, omega-functionalized alkynes having the formula HC≡C(CH₂)ₙC═CH. In addition, perfluorinated or partially-fluorinated alkyl chains, e.g., HC≡C(CF₂)ₙCF₃, can be used to lower the surface tension of hydrophobic regions to a level below that possible with non-fluorinated hydrocarbons. Alkynes can bind to surfaces such as silicon, diamond, germanium, silicon carbide, and silicon nitride through one or more C—Si, C—C, or C—N bonds.

Reactive monomers can also be used as the chemical environment to functionalize the surface. Categories of such monomers include the acrylates, methacrylates, styrenics, butadiene and derivatives and analogs thereof, maleic anhydride and maleic acid esters, vinyl ethers, acrylamide and its derivatives, monomers containing fluorinated or partially fluorinated alkyl chains, nitriles, metal salts of acrylic acid and methacrylic acid, vinylidine and vinyl monomers. Specific examples of such monomers include acrylic acid, methyl acrylate, ethyl acrylate, hydroxyethyl acrylate, butyl acrylate, lauryl acrylate, octadecyl acrylate, 2-(dimethylamino)ethyl acrylate, acryloyl chloride, methacrylic acid, methyl methacrylate, ethyl methacrylate, hydroxyethyl methacrylate, butyl methacrylate, lauryl methacrylate, octadecyl methacrylate, 2-(dimethylamino)ethyl methacrylate, methacryloyl chloride, methacrylic anhydride, monomers with more than one acrylate or methacrylate group on them, derivatives of poly(ethylene glycol) that contain one or more acrylate or methacrylate group, styrene, 2-bromostyrene, 3-bromostyrene, 4-bromostyrene, 2-chlorostyrene, 3-chlorostyrene, 4-chlorostyrene, 2-fluorostyrene, 3-fluorostyrene, 4-fluorostyrene, 4-aminostyrene, divinylbenzene, 4-styrenesulfonic acid (sodium salt), butadiene, isoprene, maleic anhydride, maleic acid, methyl vinyl ether, ethyl vinyl ether, allyl vinyl ether, dodecyl vinyl ether, octadecyl vinyl ether, acrylamide, methacrylamide, N,N-dimethylacrylamide, N-isopropylacrylamide, DuPont's Zonyl™ fluoromonomer H₂C═C(CH₃)CO₂CH₂CH₂(C—F₂)ₙ where n is about 1 to about 8, CF₃, acrylonitrile, methacrylonitrile, calcium, sodium, aluminum, silver and zirconium acrylate and methacrylate, diallyidimethylammonium chloride, vinylidine chloride, vinylidine fluoride, vinyl chloride, vinyl fluoride, itaconic acid, itaconic anhydride, cinnamic acid, cinnamoyl chloride, cinnamonitrile, esters of cinnamic acid and itaconic acid.

Combinations of two or more reactive species can be used. For example, two or more different alkenes, alkynes, or monomers can be combined. Other molecules can be added to mixtures of reactive compounds that can function as chain transfer agents in polymerizations or surfactants to keep certain species solvated. Any material that contains molecules that can react with the primarily- or subsequently-functionalized region, including gases, liquids, solids, or suspended or partially dissolved solids can be used.

In addition, compounds with two different functional groups, each of which is capable of forming a covalent bond with the surface, can be used. Specific examples include: 4-(chloromethyl)benzoylchloride, 4-(chloromethyl)benzoic acid, 3-(chloromethyl)benzoylchloride, and 4-vinylbenzyl chloride, as well as the homobifunctional and heterobifunctional binders listed hereinbelow. One functional group can react with the surface, leaving the other functional group free to react with further moieties supplied for functionalizing the surface.

The probe species can be selectively deposited on the terminally-functionalized regions of the substrate surface using any method known in the art, including microfluidic devices, microspotters or ink jet printers. Devices useful for depositing the probe species on the functionalized regions, including depositing different probe species on different functionalized regions are known to the art, e.g., as described in “DNA Arrays, Methods and Protocols,” (2001) (Jang B. Rampal, ed.), Vol. 170 of Methods in Molecular Biology, Humana Press, for example in Chapters 7 and 8. Such devices are also commercially available, for example BioRobotics, Cartesian, and GeneMachines, products available through Genomic Solutions, Ann Arbor, Mich.; QArray, QArray2, and QArrayMax) products available through Gentix, Northwich, Cheshire, England; the Nano-Plotter product available through GeSim, Groβerkmannsdorf, Germany; the NanoPrint™ and Microarrayer products available through Telechem International, Inc., Sunnyvale, Calif.; The HT-Arrayer™ product available through Bioneer USA, Rockville, Md.; the Arrayjet AJ100 product available through ArrayJet Limited, Dalkeith, Scotland; and the Xact Personal Microarrayer product available through The Gel Company, San Francisco, Calif.

Once one or more probe species have attached to the different terminally-functionalized regions, the presence of multiple target species in a sample can be tested. The target species in the sample bind with a complementary probe species on the substrate. The presence of an attached bound target species on the substrate is then detected using a technique known to the art such as a technique selected from the group consisting of fluorescence, mass spectrometry, chemosensors, matrix assisted laser desorption ionization (MALDI), mass spectrometry, time-of-flight secondary Ion Mass Spectroscopy (ToF Sims), X-ray photoelectron spectroscopy, and assays based on radioactive isotopes in target species.

Also, a probe species such as a heavy metal sensor, e.g., small organic molecules that fluoresce or cease to fluoresce when bound to heavy metals such as cadmium, mercury or lead, as known to the art, can be attached to terminally-functionalized regions, wherein the sensor species fluoresces when exposed and bound to a heavy metal [see, for example: Bull. Korean Chem. Soc. 25(6)869-872 (2004) and J. Am. Chem. Soc. 127: 16030 (2005)] can be attached to a terminally-functionalized region. The fluorescence can be detected using a chemosensor as known to the art.

Also, electroless metal deposition can also be performed on the terminally-functionalized regions. As is known to the art, electroless metal deposition involves deposit of metal without electrolysis. For example, a microcircuit can be produced by the methods of this invention by creating a pattern of functionalized lines capable of binding conductive metals on a substrate, exposing the functionalized, patterned substrate to a solution containing the desired metal to be deposited, and allowing the metal to be deposited on the functionalized lines of the substrate.

According to the present invention, the substrate can comprise a background layer coating an underlying layer. The background layer can be selected from, but is not limited to, hydrophobic materials selected from the group consisting of perfluoronated chains, fluorocarbon chains, siloxanes, alkyl chains (wherein the chains are tethered to the underlying layer), polyethylene waxes, and other hydrophobic molecules and polymers. For example, the alkyl chains can be alkyl thiols of the form CH₃(CH₂)ₙSH, and the substrate can be gold, wherein the alkyl thiol chains form a monolayer on the gold. Silanes of the form CH₃(CH₂)ₙSiCl₃ or CH₃(CH₂)ₙSi(OCH₃)₃ or other such silanes known in the art are known to bind to oxide surfaces. Alkenes of the form CH₃(CH₂)ₙCH═CH₂ and alkynes of the form CH₃(CH₂)ₙC≡CH bind to hydrogen-terminated silicon surfaces. In all of these examples n is an integer that typically has a value between 3 and 17.

The background layer can be selected in such a way to facilitate selectively depositing solutions containing probe species onto the spatially-separated functionalized regions. For example in one variation, when a pulse of laser light is directed through a lens array onto multiple regions of the substrate, the laser light changes the functionality of the substrate by melting the underlying layer while vaporizing the background layer, resulting in multiple spatially-separated primarily-functionalized regions against a hydrophobic background. The underlying layer melts but essentially no material is removed from the underlying layer. The probe species are then be selectively deposited on each of the spatially-separated functionalized regions by any method known in the art such as microspotting, or using a microfluidic device or ink jet printer. The hydrophobic background layer helps keep solutions of chemicals used for functionalization of the regions isolated from each other. For example solutions containing different probe species can be isolated to different single terminally-functionalized regions and thus the probe species is prevented from spreading onto other adjacent spatially-separated, terminally-functionalized regions. In this manner, different probe species can be attached to different terminally-functionalized regions.

The first and subsequent chemical environments do not necessarily need to be uniform across the substrate. Once portions of the background layer is removed, defining functionalized regions, liquid first and subsequent chemical environments can be selectively deposited on each of the spatially-separated functionalized regions by any method known in the art. “Selective deposit” means that a different, selected chemical environment can be deposited on different functionalized regions. The background layer keeps the liquid chemical environment confined to each of the spatially-separated primarily-functionalized regions of the substrate.

In some embodiments, however, it can be more efficient and effective to expose the whole surface to a uniform first chemical environment or to a uniform subsequent chemical environment and thus create identical spatially-separated functionalized regions. For example, it may be desirable to have the secondarily-functionalized spatially-separated regions contain identical functional groups. In such a case, each of the primarily-functionalized regions are exposed to a uniform first chemical environment and allowed to react, creating secondarily-functionalized regions with identical functional groups. It may then be desired to have tertiarily-functionalized regions that have differing functional groups. A nonuniform second chemical environment would then be selectively deposited on each of the secondarily-functionalized regions, and allowed to react, creating tertiarily-functionalized regions with differing functional groups.

Thus, differing chemical reactions may be performed and functional groups built up on each of the separate spatially-separate functionalized regions until desired terminally-functionalized regions are achieved. Then different types of probe species can be deposited on the separate spatially-separated terminally-functionalized regions. For example, in order to attach a protein probe species having cysteine residues with thiols to a terminally-functionalized region, the terminally functionalized region would need a terminally-functionalized region with a thiol-reactive group such as a maleimide. On the other hand, in order to attach an amine-terminated oligonucleotide probe species to a terminally-functionalized region, the terminally-functionalized region would need an amine-reactive group such as an NHS ester.

The background layer is preferably a thin film or monolayer which can be deposited on the substrate using methods known to one of skill in the art, including methods selected from the group consisting of chemical reactions between functional groups on the substrate and functional groups in a molecule that will be tethered to the substrate, spin coating, melting, dip coating, evaporation, plasma polymerization, and spraying. For example, a hydrophobic monolayer can be made on silicon using the following process: A hydrogen-terminated silicon wafer is prepared using a 5 percent hydrofluoride (HF) etch for approximately ten minutes. The hydrogen-terminated silicon wafer is then immersed in a refluxing solution of one 1-hexadecene in mesitylene for a few hours to produce a hydrophobic alkyl monolayer on silicon.

If using fluorescence to detect target species, the ability to detect the presence of an attached bound target species can be increased by incorporating a fluorescence-quenching compound into the background layer. When exposed regions of a substrate are functionalized by a pulse of laser light passing through a microlens array, the pulse of laser light removes portions of the background layer including the fluorescence quenching compound at the exposed regions, resulting in multiple spatially-separated primarily-functionalized regions against a fluorescence-quenching background layer. The fluorescence quenching background layer quenches the fluorescence of any fluorescing species that may randomly adsorb to the background layer when testing for target species. For example, 2,2,6,6-tetramethyl-1-piperidinyloxy, free radical (TEMPO) or other molecules that contain permanent radicals, can be incorporated into the background layer to quench fluorescence. Such molecules can be destroyed by laser light. Heavy atoms such as iodine can also be used in the background layer to quench fluorescence, but such atoms are more difficult to remove with a laser than organic compounds that can be broken down by laser light.

The details of one or more embodiments of the invention are set forth in the accompanying drawing and the description below. Other features, objects, and advantages, of the invention will be apparent from the description and drawings, and from the claims. The embodiments described below are meant to be illustrative and not limiting in any way.

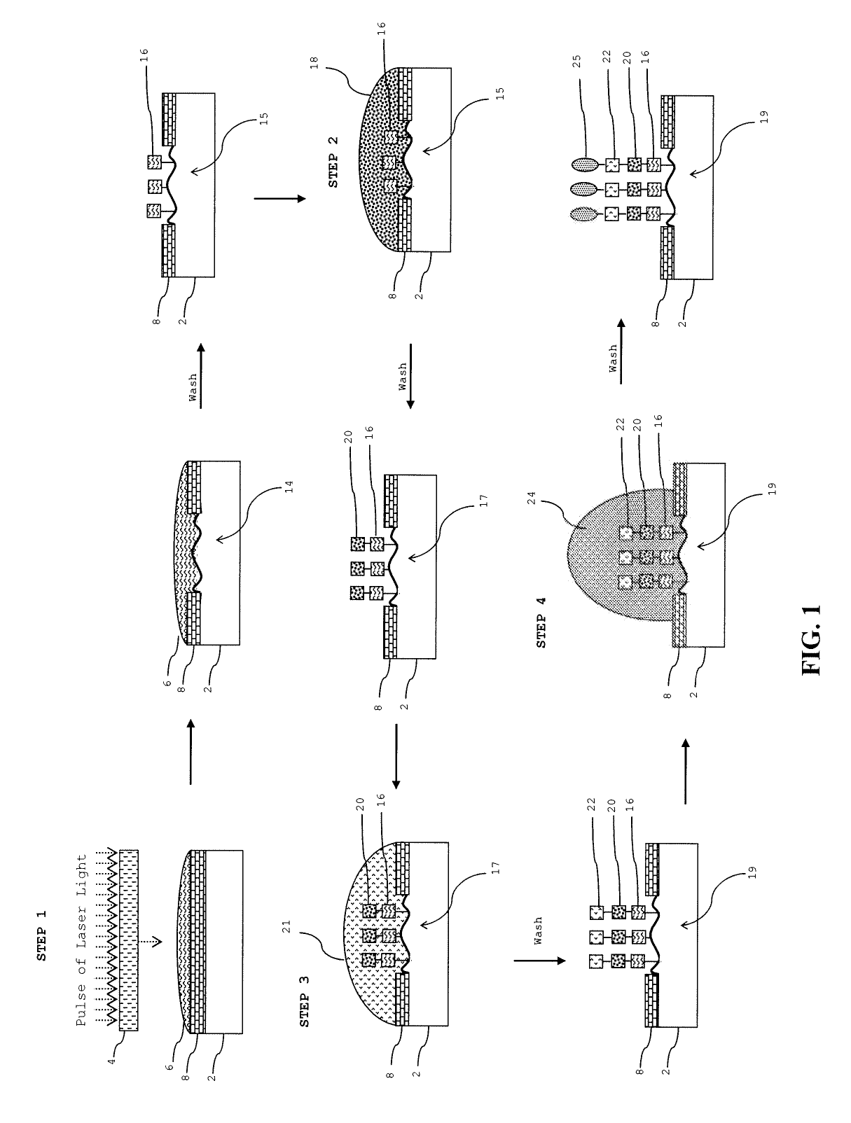

Referring to FIG. 1, the substrate comprises a background layer 8 and an underlying layer 2. The underlying layer 2 is coated with a background layer 8 that is a hydrophobic thin film or monolayer.

In Step 1 a pulse of laser light is directed through a lens array that focuses the laser light onto a region of the substrate. FIG. 1 depicts the pulse of laser light being focused by a single lens 4 of a lens array onto a single region of the substrate. The other regions (not shown) on the substrate that are exposed to the pulse of laser light simultaneously undergo essentially the same process depicted in FIG. 1. The pulse of laser light passes through a first chemical environment onto the surface of the substrate. The chemical environment comprises ambient air (not labeled). The pulse of laser light then passes through a liquid, chemical environment 6, which can be an alcohol, for example.

The pulse of laser light changes the functionality of the region of the substrate on which it falls, resulting in a primarily-functionalized region spatially separated, substantially spatially separated, from other primarily-functionalized regions where light focused by the other lenses of the lens array falls. In FIG. 1, the laser light changes the functionality of the substrate by melting underlying layer 2 (shown by the wavy line) while removing a portion of background layer 8, resulting in a primarily-functionalized region 14. The pulse of laser light is of a duration and energy required to heat and melt the surface of the underlying layer and cause a portion of the background layer to be removed while avoiding loss of material from the underlying layer. The heat from this near-instantaneous melting of the underlying layer removes the thin background layer 8 (which can be a monolayer or thin film), leaving exposed highly-reactive species of the underlying substrate, resulting in a primarily-functionalized region 14.

The primarily-functionalized region 14 immediately reacts with the liquid (alcohol) chemical environment 6 creating a secondarily-functionalized region 15 with second functional groups 16. The substrate underlying layer 2 is washed leaving exposed the secondarily-functionalized region 15 with second functional groups 16. In this embodiment, the second functional groups 16 include hydroxyl functional groups.

In step 2, the substrate is exposed to a second chemical environment 18 which supplies a third functional group 20 such as an amine group. Other suitable third functional groups 20 can be selected from the group consisting of one or more homobifunctional or heterobifunctional binders. Homobifunctional binders (crosslinkers) have two identical reactive groups and are useful in one-step chemical crosslinking procedures. Homobifunctional binders useful herein include cadaverene (1,5-diaminopentane), putrescine (1,4-diaminobutane), any other diamine, 1,4-butanediol diglycidyl ether, bisphenol A diglycidyl ether, polyethylene glycol diglycidyl ether, any other diepoxide, 1,4-phenylene diisocyanate, 1,6-diisocyanatohexane, any other diisocyanate, 1,4-phenylene diisothiocyanate, tolylene-2,4-diisothiocyanate, any other diisothiocyanate, adipoyl chloride, any other diacid chloride, ethylene glycol bis[succinimidylsuccinate], bis[2-(succinimidooxycarbonyloxy)ethyl]sulfone, dithiobis[succinimidylpropionate], disuccinimidyl tartarate (a crosslinker that can be subsequently cleaved by oxidizing reagents), disuccinimidyl suberate (this crosslinker cannot be subsequently cleaved), any other molecule with two N-hydroxysuccinimide (NHS) ester groups, ethylene glycol bis[sulfosuccinimidylsuccinate], any other molecule with two sulfo-NHS ester groups (sulfo-NHS ester groups impart greater water solubility to compounds than NHS ester groups), 1,4-di(maleimido)butane, N,N-(1,4-phenylene)dimaleimide, any other molecule with two maleimide groups, 1,5-difluoro-2,4-dinitrobenzene, any other molecule with two or more reactive fluorines on an aromatic ring, 1,6-Hexane-bis-vinylsulfone (this molecule can be used to couple sulfhydryl groups without the risk of subsequent hydrolysis), any other molecule with two vinyl sulfone groups, 1,4-di-[3′-(2′-pyridyidithio)-propionamido]butane. Heterobifunctional binders (crosslinkers) have two identical reactive groups and allow sequential conjugations, minimizing polymerization. Heterobifunctional binders useful herein include 4-(4-maleimidophenyl)butyric acid N-hydroxysuccinimide ester (a maleimide group coupled to an NHS ester), Succinimidyl-4-[N-Maleimidomethyl]cyclohexane-1-carboxy-[6-amidocaproate] (another maleimide group coupled to an NHS ester), N-e-Maleimidocaproyloxy]sulfosuccinimide ester (a sulfo-NHS ester coupled to a maleimide), (succinimidyl 4-formylbenzoate) (an NHS ester coupled to an aldehyde), 4-succinimidyloxycarbonyl-methyl-a-[2-pyridyidithio]toluene (can be used to couple amino groups with the NHS ester and sulfhydryl groups with the pyridyidithio groups). These homo- and heterobifunctional binders can also contain active halogen groups, e.g., —C(O)—CH₂—Br or —C(O)—CH₂—I, which react with sulfhydryl and amino groups. Any homo- or heterobifunctional compounds known to the art can be used in this invention, e.g., as described in G. Hermanson, (1996), “Bioconjugate Techniques,” Academic Press.

The third functional groups 20 are allowed to react with the second functional groups 16 on secondarily-functionalized region 15. The second chemical environment 18 is washed from the surface leaving exposed a tertiarily-functionalized region 17 with third functional groups 20 bound to the second functional groups 16.

In Step 3, the substrate is exposed to a third chemical environment 21 that includes fourth functional groups 22 such as homo- or heterobifunctional crosslinkers as described above. The fourth functional groups 22 are allowed to react with the third functional groups 20 of the tertiarily-functionalized region 17. The third chemical environment 21 is then washed from the surface leaving exposed a quaternarily-functionalized region 19 with fourth functional groups 22. In this embodiment the quaternarily-functionalized region is the terminally-functionalized region.

In Step 4, the probe species 25 are attached to the fourth functional groups 22 on the quaternarily-functionalized region 19 by depositing a solution 24 containing the probe species 25 on the quaternarily-functionalized region 19. In order to attach different probes to different terminally-functionalized regions, the solution that contains the probe species 25 can be selectively deposited on the quaternarily-functionalized region 19 by any method known in the art, including using a microfluidic device, an ink jet printer, or a microspotter. The term “selectively deposited” means that different selected probes can be deposited in different selected functionalized regions on the substrate, or that different selected probes can be deposited on the functional groups present on a single region. The probe species 25 are allowed to react with the quaternarily-functionalized region 19. The solution 24 is then washed from the surface leaving exposed the probe species 25.

In another embodiment, the liquid chemical environments are selected such that probes can be added during Step 3 instead of Step 4 of FIG. 1. For example, the liquid chemical environment 6 can comprise cadavarene, and the pulse of laser light causes a reaction whereby the second functional groups 16 attached to region 14 are amine groups. In step 2, the liquid chemical environment 18 could comprise homobifunctional crosslinkers as described above, which react with the probe species, in a third step, thereby attaching the probe species to the substrate.

In yet another embodiment, the liquid chemical environments are selected such that probe can be added during Step 2 instead of Step 4 of FIG. 1. In this embodiment, Step 1 produces a secondarily-functionalized region 15 that is the terminally-functionalized region. For example, when epoxy octane is selected as a liquid chemical environment 6, Step 1 produces a secondarily-functionalized region 15 with functional groups 16 comprising epoxide groups. In Step 2, the probe species are attached to the secondarily-functionalized region 15 by depositing a solution containing the probe species on the secondarily-functionalized region.

In another embodiment of the present invention liquid chemical environment 6 is deposited subsequent to creating the primarily-functionalized region. In other words, the substrate is not exposed to the liquid chemical environment simultaneous with directing the light through the microlens array. In this embodiment, the first step is performed in a vacuum, and the pulse of laser light functionalizes a region of the substrate simply by removing the background layer in the region where it falls, or by producing reactive species on the surface of the underlying layer. The underlying layer can be melted or partially melted, or in other embodiments, is not melted. In a second step, a first liquid chemical environment comprising an aqueous solution, e.g., polylysine, is deposited on the primarily-functionalized region and the polylysine reacts with the primarily-functionalized region creating a secondarily-functionalized region with second functional groups comprising primary amine groups. Then, in a third step, the substrate is exposed to a second liquid chemical environment which includes third functional groups that react with the primary amines, e.g., homo- or heterobifunctional crosslinkers. The third functional groups are allowed to react with the second functional groups on the functionalized region. The second chemical environment is then washed from the surface leaving exposed a tertiarily-functionalized region with third functional groups bound to the second functional groups. In a fourth step, the substrate is exposed to a third liquid chemical environment that includes fourth functional groups such as homo- or heterobifunctional crosslinkers. The fourth functional groups are allowed to react with the tertiarily-functionalized region. The third chemical environment is then washed from the surface leaving exposed a quaternarily-functionalized region with fourth functional groups. In this embodiment the quaternarily-functionalized region is the terminally functionalized region, i.e., the region capable of reacting with the probe species to attach the probe species to the substrate through the intervening functional groups. This is done in a fifth step by depositing a solution containing the probe species on the quaternarily-functionalized (terminally-functionalized) region. The solution that contains the probe species can be deposited on the quaternarily-functionalized region by any method known in the art, including using a microfluidic device, an ink jet printer or a microspotter. The solution can then be washed from the surface leaving the probe species exposed. Different probe species can be attached to different regions, or a single region can have one, two, three, or more probe species attached to the functional groups in that region.

If desired, the substrate can be prepared without the background layer. For example, if a silicon wafer is chosen as the substrate, the silicon wafer can be cleaned by a method known in the art, which leaves behind or substantially leaves behind the native oxide layer of silicon oxide on the silicon. As another example, if the substrate is polymethylmethacrylate, the surface can first be cleaned by a method known in the art, e.g., by soap and water with sonication, and then rinsed with pure water and dried. The first chemical atmosphere is then applied to the substrate without a background layer.

In certain variations of the invention, a solution containing only a single probe species can be used, and the probe species is attached to a single terminally-functionalized region. Thus, the functionalized substrate could only test for a single target species. In another variation, a single terminally-functionalized region can be used to test for multiple target species. According to this variation, a solution applied to a terminally-functionalized region can comprise multiple different probe species such that multiple probe species are attached to a single terminally-functionalized region. Fluorescence or mass spectrometry or other detection means known to the art could then be used to determine the presence of different target species bound to different probe species on a single terminally-functionalized region. In this way, a greater number of target species can be tested per unit area of substrate.

In yet another variation, the surface area of the functionalized regions can be increased on a nano-scale without changing the overall shape or diameter of the exposed regions. The increase in surface area increases the density of functional groups that can be reacted with the functionalized region, which increases the density of the probe species that can be attached to the terminally-functionalized region without increasing the diameter or overall shape of the functionalized region. For example, silanol-terminated silicon nanoparticles can be mixed with an aqueous solution of polylysine. The polylysine changes the functionality of the surface of the nanoparticles by adsorbing at their surfaces such that when the polylysine-coated nanoparticles are deposited on the functionalized regions, e.g., on the primarily-functionalized regions, the nanoparticles increase the roughness of the surface, thereby increasing the surface area of the regions, and provide a greater number of reactive amine groups for subsequent coupling of probe species.

In another variation, two pulses of laser light can be used to increase the density of the primarily-functionalized regions. The first pulse of laser light is directed onto the substrate through a microlens array to create multiple primarily-functionalized regions. The substrate is then repositioned such that a second pulse of laser light passes through the microlens array onto regions of the substrate separate from the first primarily-functionalized regions. The second pulse of laser light creates additional spatially-separated primarily-functionalized regions that are spatially-separated from the first primarily-functionalized regions. In this manner the density of primarily-functionalized regions on the substrate can be increased.

### EXAMPLES

### Example 1

In this example we show that we can wet a semiconductor surface, e.g., silicon or germanium, with a reactive compound and then fire a highly focused, nanosecond pulse of laser light through the transparent liquid onto the surface. The high peak power of the pulse at the surface activates the surface so that it reacts with the liquid it is in contact with. This work was performed with single lenses, not microlens arrays. Similar and sometimes identical chemistry occurs using microlens arrays. Unless otherwise indicated experimental conditions for the results in this example are as follows: 532 nm light from a Coherent Infinity Nd:YAG laser, with a 4 ns pulse width, 50-100 μJ pulse energy, and the calculated diameter of the laser at the surface is 50-100 μm. Average values and errors (standard deviations) in this work are from three measurements.

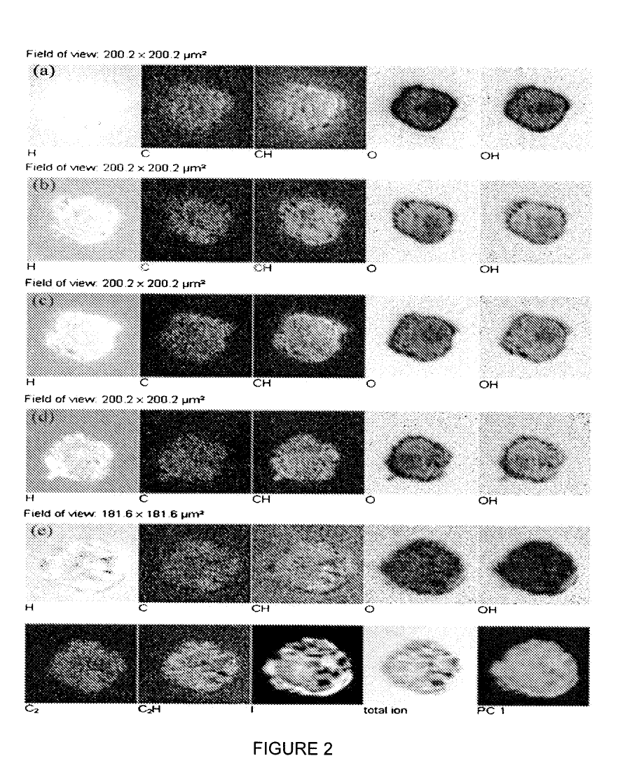

FIG. 2 shows representative time-of-flight secondary ion mass spectrometry (ToF-SIMS, ION-TOF IV) negative ion images of spots of Si that was wet with 1-hexene, 1-decene, 1-tetradecene, and octane.

FIG. 2e shows ToF-SIMS negative ion images of a clean germanium surface that was wet with 1-iodooctane. The chemical contrast evident in these images is consistent with chemical modification of the silicon and germanium in the laser spots with the hydrocarbon compounds. These results are quite general. Similar images, with similar chemical contrast between functionalized spots and backgrounds are found for silicon wet with 1-octene, 1-dodecene, 1-hexadecene, 1-chlorooctane, 1-bromooctane, 1-iodooctane, 1-octanol, 1,2-epoxyoctane, and 1,2,7,8-diepoxyoctane. ToF-SIMS shows the expected halogen ions from the haloalkanes, as in FIG. 2e. ToF-SIMS ion images of laser spots of germanium wet with 1-hexadecene also show similar chemical contrast between spots and background regions as is found in FIG. 2.

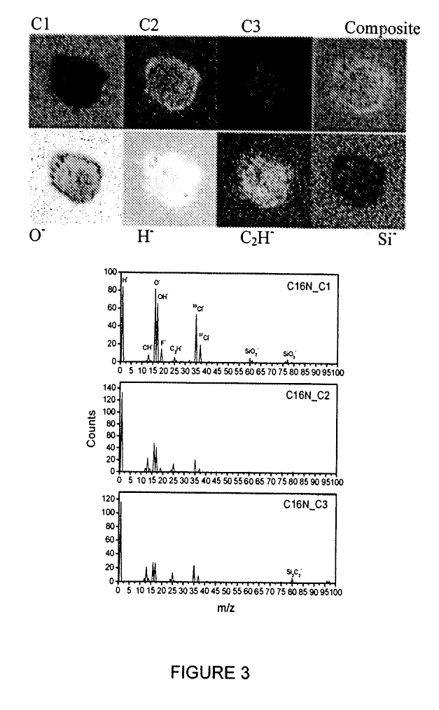

To better understand the chemical nature of the variation in the spectral images shown in FIG. 3, a multivariate analysis of the data was performed using the Automated eXpert Spectral Image Analysis (AXSIA) method (Ohlhausen, J. A. et al. (2004) Appl. Surf. Sci. 231-232, 230-234; Smentkowski, V. S. et al. (2004) Appl. Surf. Sci., 231-232, 245-249). AXSIA reduces ToF-SIMS images into a limited number of components that sufficiently describe the chemical variation at a surface; AXSIA components better represent the chemical information at a sample surface than individual ToF-SIMS images of single ions.

FIG. 3 shows Negative-ion AXSIA spectral images, a composite image of AXSIA components 1-3 (C1-C3), and single ion images of ToF-SIMS of silicon surfaces modified with a laser using 1-decene (top images). Spectra of AXSIA components of functionalized spots on silicon modified with 1-hexadecene (bottom spectra). This Figure shows a few ion images from a laser spot and background made on silicon that was wet with 1-decene, images of the AXSIA components in red, green, and blue that were derived from an AXSIA analysis of this ToF-SIMS data, and some AXSIA spectral components from a spot made with 1-hexadecene. These results are quite general. A large number of ToF-SIMS images of spots made with different hydrocarbon reagents were analyzed by AXSIA. AXSIA almost always finds three components. One component corresponds to the background, away from the functionalized spot, that is rich in O⁻, OH⁻, F⁻, and Cl⁻, and that also contains small SiO₂⁻ and SiO₃⁻ ions. The two other AXSIA components are usually quite similar and come from the functionalized spot. These two components contain a larger fraction of H⁻ and CH⁻ ions than the background component, and less oxygen-containing ions and halogen contaminants. Note that ToF-SIMS is very sensitive to trace halogens—XPS shows that chlorine and fluorine contamination at and around functionalized spots is at very low levels (vide infra). The upshot of these results is that, although the matrix effect of SIMS prevents quantification by direct comparison between peaks, ToF-SIMS reveals chemical variation between the spots and their backgrounds that is consistent with hydrocarbon functionalization in the spots. As noted, this analysis suggests increased levels of hydrogen in the spots, which is valuable information that cannot be obtained by XPS.

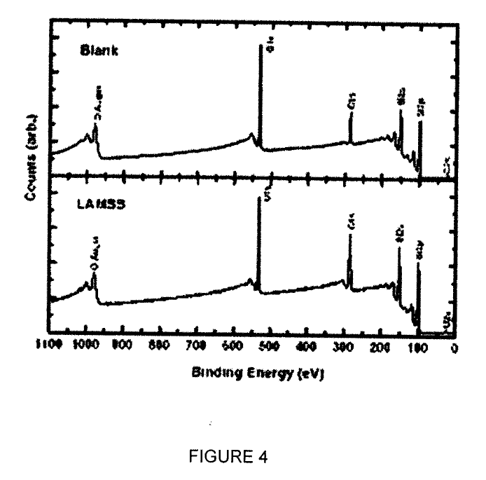

X-ray photoelectron spectroscopy (XPS) was also used to probe the elemental composition of functionalized spots and control regions on silicon. FIG. 4 shows XPS survey spectra of a) a blank region on a silicon surface that had been wet with 1-hexadecene, but not exposed to a pulse of laser light, and b) a functionalized laser spot made with 1-hexadecene. The control region shows strong oxygen, carbon, and silicon signals, where the carbon in this spectrum is presumably due to adventitious material. The survey spectrum from the functionalized spot in FIG. 4b also shows primarily oxygen, carbon, and silicon, but the elements appear to exist in different ratios than the control region; the oxygen signal appears somewhat reduced and the carbon signal increased. The C1s/Si2p and O1s/Si2p ratios of these surfaces, of a functionalized spot and of an adjacent control made with 1-iodooctane, and the C1s/Si2p ratio of an alkyl monolayer on hydrogen-terminated silicon given for comparison, are provided in Table 1. It is noteworthy in these results that the C1s/Si2p ratio for the functionalized spot prepared under 1-hexadecene is similar to the C1s/Si2p ratio obtained from a 1-hexadecene monolayer on hydrogen-terminated silicon, and that the O1 s/Si2p ratios for the functionalized spots are smaller than the ratios found in control regions.

TABLE 1
C1s/Si2p and O1s/Si2p XPS ratios of functionalized laser spots
and control regions

| | C1s/Si2p | O1s/Si2p |
|---|---|---|
| Surface composition | ratio | ratio |
| 1-hexadecene | 1.12 ± 0.05²² | 0.66 ± 0.02 |
| 1-hexadecene control | 0.67 ± 0.03¹³ | 0.87 ± 0.01 |
| 1-iodooctane | 0.66 ± 0.05 | 0.64 ± 0.03 |
| 1-iodooctane control | 0.23 ± 0.02¹³ | 0.84 ± 0.01 |
| 1-hexadecene on hydrogen terminated | 1.27 ± 0.03 | |
| silicon. 16 Å. | | |

*Literature control. Reference: Yang, L.; Lua, Y.—Y.; Lee, M. V.; Linford, M. R. Acc. Chem. Res. 2005, 38, 933-942.

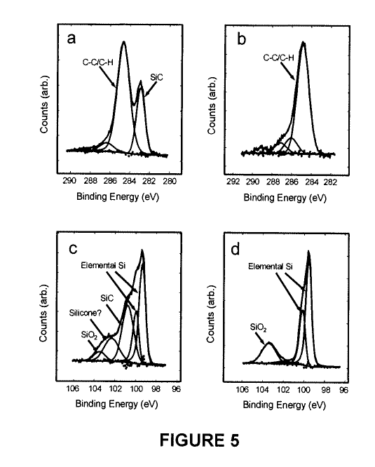

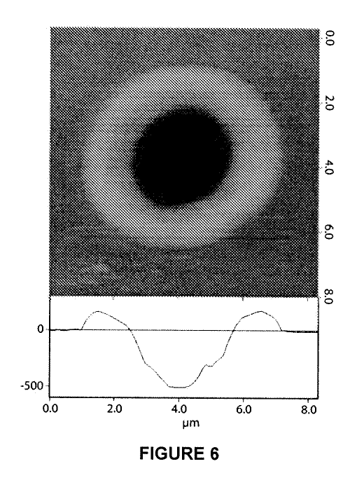

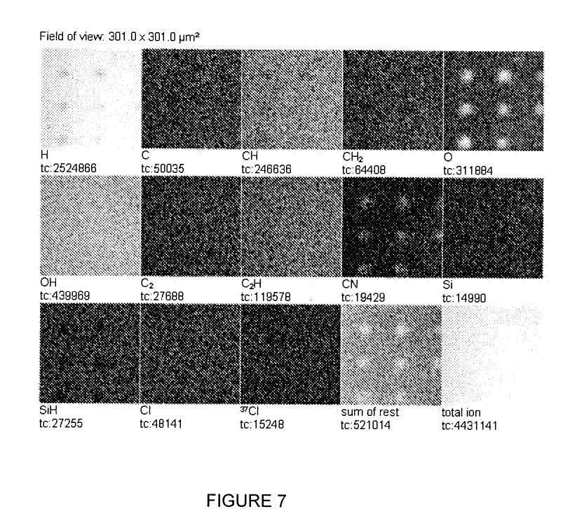

XPS narrow scans provide additional information about control and functionalized laser spots; FIG. 5 shows XPS narrow scans of the C1s and Si2p regions that corresponds to the survey spectra shown in FIG. 3, and that indicate the oxidation states of the carbon and silicon atoms at the surfaces. The C1s narrow scan of the control region (FIG. 5b) shows mostly carbon bonded to carbon and hydrogen. In contrast, the C1s narrow scan of the functionalized spot (FIG. 5a) consists primarily of two peaks: a larger signal that corresponds to carbon bonded to carbon and hydrogen, and a smaller, but still very significant peak that we identify as silicon carbide. The Si2p narrow scans are consistent with the C1 s results. The control region is almost entirely composed of signals from bulk silicon and oxide. In contrast, the Si 2p narrow scan of the functionalized spot shows many oxidation states for silicon, including silicon carbide, and a silicone-like species, i.e., silicon bonded to both oxygen and carbon atoms. Table 2 contains a deconvolution of the C1s and Si2p regions for functionalized spots made with 1-hexadecene and 1-iodooctane, and corresponding controls. It is significant that strong hydrocarbon and silicon carbide signals are observed in the functionalized spots, but only hydrocarbon signals are present in the controls.

TABLE 2
Deconvolutions of C1s and Si2p narrow scans from
functionalized spots and control regions.

| | | | 1- | | |
|---|---|---|---|---|---|
| | | 1- | hexadecene | 1- | 1-iodooctane |
| Sample | | hexadecene | control | iodooctane | control |
| % Carbon | Si—C | 26.43 ± 1.89 | — | 47.7 ∓ 1.21 | — |
| [symbol] | (carbide) | | | | |
| | C—C, C—H | 67.7 ± 2.43 | 83.65 ± 1.77 | 46.3 ± 2.12 | 78.2 ± 1.27 |
| | C—O | 4.43 ± 1.27 | 8.3 ± 0.14 | 4.03 ± 0.60 | 10.3 ± 1.41 |
| | C═O | — | 1.7 ± 0.42 | — | 7.3 ± 0.85 |
| | O—C═O | 1.47 ± 0.40 | 6.4 ± 1.56 | 1.93 ± 0.40 | 4.2 ± 0.85 |
| % Silicon seen as | Elemental | 47.73 ± 1.99 | 75.3 ± 0.42 | 47.9 ± 3.02 | 75.65 ± 0.64 |
| | Si | | | | |
| | Si—C | 30.57 ± 1.46 | — | 28.3 ± 2.85 | — |
| | (carbide) | | | | |
| | Silicone (?) | 16 ± 0.26 | 3.3 ± 0.14 | 10.27 ± 0.32 | 3.3 ± 0.85 |
| | SiO₂ | 5.73 ± 1.15 | 21.45 ± 0.49 | 13.57 ± 0.32 | 21.05 ± 0.21 |

Values in this table are the average of three measurements on three different spots. Errors are the standard deviations of these three values.

These XPS and ToF-SIMS results make it clear that high energy laser pulses can drive surface reactions that would not be possible at room temperature. In spite of this, it appears that some chemical functionality is preserved in the laser functionalization process. For example, functionalized spots were made in the air, and on silicon surfaces wet with octane, 1-octene, and 1,7-octadiene. After formation of these functionalized spots, the surfaces were exposed to HCl vapor. HCl readily reacts with carbon-carbon double bonds. The following ToF-SIMS Cl/Si ratios were calculated for the resulting functionalized spots: 0.23±0.04 (air), 6.4±1.8 (octane), 5.8±2.3 (1-octene), and 14.7±1.4 (1,7-octadiene). (The Cl/Si ToF-SIMS ratio is the ratio of peak areas from the negative ion spectra as follows: (35Cl+37Cl)/(SiO²+SiHO²+SiO³+SiHO³+SiHO+Si⁺ SiH).) These results are consistent with retention of functionality from the diene, and creation of double bonds by thermal cracking of the alkane and alkenes.

In many applications it would be advantageous to have smaller spots than the ca. 150 □m spots shown in FIG. 1. FIG. 5 shows a functionalized feature made with a 25 mm focal length achromat doublet lens. The spots were produced with 5 μJ of energy per pulse, which corresponds to a peak laser power of 5×10⁹ W/cm². The diameter of the feature is 6 μm, with a noticeable raised ring around a 4 μm diameter, 500 nm deep spot. An atomic force microscopy (AFM) analysis of sub-10 μm functionalized spots made in this manner suggests that the volume of material above the plane of the substrate is roughly equal to the volume of the recessed region below the plane. In other words, the focused, low-power laser shots appear to primarily cause surface melting (the m.p. of Si is 1414° C.), rather than ablation, although ablation is clearly seen in functionalized regions at higher laser powers. The high temperatures that must be present at the point of the functionalized spot during activation is well above that needed to crack hydrocarbons (450-750° C.). This would help explain the reactivity of an alkane (octane) and carbide/silicone formation at the surface.

### Example 2

Sample Cleaning. Pieces of silicon were cleaned with a 2% solution of sodium dodecyl sulfate (a surfactant). They were then plasma cleaned in a Harrick plasma cleaner (5 minutes on high power).

Hydrophobic Monolayer Formation: After this second cleaning, the surfaces were put in a 5% aqueous solution of HF for 8 minutes to remove the native oxide layer on the silicon surface and leave behind hydrogen-terminated silicon. This hydrogen-terminated silicon was then immersed in neat (pure) 1-hexadecene (≧99.0), which had been degassed by bubbling nitrogen gas through it for an hour. After the silicon was put in the 1-hexadecene the liquid was further degassed by bubbling with nitrogen for at least another 0.5 hr. The pure 1-hexadecene with the silicon shard in it was then heated to 210° C. for 1 hour.

Sample Cleaning: The silicon samples were then removed and washed several times with hexane and ethanol. The surfaces were further cleaned by sonication twice in methylene chloride (CH₂Cl₂) for five minutes. The surfaces were finally washed with ethanol and dried with nitrogen gas.

Making Laser Spots: A pulse of 532 nm laser light was directed through a lens array onto the 1-hexadecene monolayer-coated silicon surface. The medium between the lens array and the monolayer-coated silicon surface was ambient air.

Polylysine Adsorption: The silicon surfaces were then immersed in a 0.01% (w/v) aqueous solution of polylysine for 1 hr. After this immersion the samples were removed and rinsed with DI water and finally dried with nitrogen gas.

Characterization: Time-of-flight secondary ion mass spectrometry confirmed the presence of polylysine localized in spots made by a lens array in the form of CN⁻ ions from the spots. See FIG. 7.

Further Modification: Through methods known in the art, amine-terminated oligonucleotides or proteins, or a variety of other species, can then be covalently attached to the polylysine spots that were created in this manner. Oligonucleotides or appropriate proteins can also electrostatically adsorb onto the polylysine surface, by methods known in the art.

A number of embodiments of the invention have been described. Nevertheless, it will be understood that various modifications can be made without departing from the scope of the invention. Accordingly, other embodiments are within the scope of the following claims.

1. A method of functionalizing a substrate comprising:
(a) providing a substrate; and
(b) directing light through a microlens array onto said substrate;
whereby said microlens array focuses said light onto multiple spatially-separated regions of said substrate; and wherein said light is of sufficient energy to change the functionality of the multiple regions, resulting in primarily-functionalized regions that are spatially separated.

2. The method of claim 1 further comprising:
(c) exposing the primarily-functionalized regions to a first chemical environment which causes the primarily-functionalized regions to undergo a secondary change in functionality resulting in multiple secondarily-functionalized regions;
(d) removing the first chemical environment leaving exposed the multiple secondarily-functionalized regions;
(e) optionally repeating steps (c) and (d) with second and subsequent chemical environments until desired multiple terminally-functionalized regions are achieved; and
(f) attaching at least one probe species to the terminally-functionalized regions.

3. The method of claim 2 wherein the substrate is exposed to the first chemical environment simultaneous with directing the light through the microlens array.

4. The method of claim 1 wherein the light causes the change in functionality of the multiple regions through at least one of ablation, melting, activation, and photo-cleavage.

5. The method of claim 1 wherein the substrate is in a vacuum.

6. The method of claim 1 wherein the substrate comprises at least one material selected from the group consisting of silicon, diamond, fused silica, glass, germanium, silane monolayer, alkene monolayer, thiol monolayer, Teflon™, metal, polyelectrolyte film, diamond, silicon nitride, silicon carbide, polycarbonate, polydimethylsiloxane, and polymethylmethacrylate.

7. The method of claim 1 wherein the substrate comprises a background layer and an underlying layer, wherein the background layer coats the underlying layer and wherein the background layer is selected from the group consisting of materials comprising perfluoronated chains, fluorocarbon chains, siloxanes, alkyl chains, functionalized alkyl chains, polyethylene waxes, and polyethylene glycol.

8. The method of claim 7 further comprising coating the underlying layer with the background layer before exposing the multiple regions of the substrate to the light, wherein the light functionalizes regions of the substrate by removing portions of the background layer.

9. The method of claim 2 wherein the terminally-functionalized regions comprise chemically-reactive moieties selected from the group consisting of at least one of amine groups, alcohol groups, epoxide groups, N-hydroxysuccinimide (NHS) ester groups, acid chloride groups, isothiocyanate groups, isocyanate groups, carboxyl groups, vinyl sulfone groups, fluorine-functionalized aromatic rings, aldehyde groups, alkyl halide groups, sulfonyl chloride groups, maleimide groups, benzyl halide groups, aromatic rings, carbon-carbon double bonds, carbon-carbon triple bonds, methyl esters, carbodiimides, and acid anhydride groups.

10. The method of claim 1 wherein the light is a laser light.

11. The method of claim 1 wherein the light passes through the microlens array and then passes through the first chemical environment, which comprises ambient air in contact with said microlens array and a liquid in contact with said substrate.

12. The method of claim 1 wherein the microlens array is in direct contact with a liquid chemical environment.

13. The method of claim 2 wherein the first chemical environment comprises a gas selected from the group consisting of at least one of ambient air, nitrogen, oxygen, argon, helium, ethylene, acetylene, butene, methane, and butane.

14. The method of claim 2 wherein the first chemical environment comprises a solid selected from the group consisting of at least one of polystyrene, polymethylmethacrylate, polytetrafluoroethylene, alkyl monolayer, hydrocarbon wax, and polydimethylsiloxane.

15. The method of claim 2 wherein the first chemical environment comprises a liquid comprising at least one compound selected from the group consisting of water, alcohol, compounds supplying functional groups selected from the group consisting of hydroxy groups, amine groups, alkyl halide groups, alkynes, carbon disulfides, epoxide groups, carboxylic acid groups, compounds having at least one aromatic ring, alkenes, NHS esters, acid chlorides, acid anhydrides, methyl esters, isocyanates, isothiocyanates, vinyl sulfones, fluorine-functionalized aromatic rings, aldehydes, carbodiimides, benzyl halides, carbon disulfide, epoxides, carboxylic acids, thiols, halides, aldehydes, ketones, amides, carboxylic acid esters, acrylates, methacrylates, vinyl ethers, acrylamides, azides, nitrites, dienes, trienes, phosphines, isocyanates, isothiocyanates, silanols, oximes, diazo, epoxides, nitro groups, sulfate groups, sulfonate groups, phosphate groups, phosphonate groups, anhydride groups, guanadino groups, phenolic groups, imines, diols, triols, hydrazones, hydrazines, disulfide groups, sulfide groups, sulfone groups, sulfoxide groups, peroxide groups, urea groups, thiourea groups, carbamate groups, diazonium groups, azo groups, DNA, RNA, protein, carbohydrates, lipids, and styrenics.

16. The method of claim 2 in which electroless metal deposition is performed on the terminally-functionalized regions.

17. The method of claim 2 wherein the at least one probe species is selected from the group consisting of polypeptides, antibodies, proteins, enzymes, nucleic acids, oligosaccharides, polyamide nucleic acids, and fluorescent chemosensors.

18. The method of claim 2 wherein the at least one probe species is attached to the terminally-functionalized region using means selected from the group consisting of microfluidic devices, microspotters, and ink jet printers.

19. The method of claim 2 wherein multiple probe species are attached to at least one of the terminally-functionalized regions.

20. The method of claim 2 further comprising determining whether the target species has bound to the probe species using a method selected from the group consisting of fluorescence, mass spectrometry, chemosensing, matrix assisted laser desorption ionization (MALDI), mass spectrometry, time-of-flight secondary Ion Mass Spectroscopy (ToF Sims), X-ray photoelectron spectroscopy, and assays based on radioactive isotopes in target species.

21. A method of functionalizing a surface comprising:
(a) providing a substrate;
(b) directing light onto the substrate wherein the light melts material of the substrate without causing measurable loss of material therefrom other than material of any background layer of the substrate, thereby creating a primarily-functionalized region; and
(c) exposing the primarily-functionalized region to a first chemical environment which causes the primarily-functionalized region to undergo a secondary change in functionality resulting in a secondarily-functionalized region.

22. The method of claim 21 further comprising:
(d) removing the first chemical environment, leaving exposed the secondarily-functionalized region;
(e) optionally exposing the secondarily-functionalized region to a subsequent chemical environment and removing the subsequent chemical environment, and optionally repeating this process with tertiarily-functionalized regions and further-functionalized regions until a terminally-functionalized region is achieved; and
(f) attaching at least one probe species to the secondarily-functionalized region or the terminally-functionalized region.

23. The method of claim 21 wherein the substrate is exposed to the first chemical environment simultaneous with directing the light onto the exposed region.

24. The method of claim 21 wherein the light is directed onto the substrate through a microlens array.

25. The method of claim 22 wherein the terminally-functionalized region chemically-reactive moieties selected from the group consisting of at least one of amine groups, alcohol groups, epoxide groups, N-hydroxysuccinimide (NHS) ester groups, acid chloride groups, isothiocyanate groups, isocyanate groups, carboxyl groups, vinyl sulfone groups, fluorine-functionalized aromatic rings, aldehyde groups, alkyl halide groups, maleimide groups, sulfonyl chloride groups, benzyl halide groups, aromatic rings, carbon-carbon double bonds, carbon-carbon triple bonds, methyl esters, carbodiimides, and acid anhydride groups

26. The method of claim 21 further comprising preparing the substrate by coating an underlying layer with a background layer before exposing the multiple regions of the substrate to the light, wherein the light removes the background layer while melting regions of the underlying layer.

27. The method of claim 21 in which the light is laser light.

28. A system for making an assay device comprising:
(a) a laser capable of delivering a pulse of laser light having an energy between about between about 10⁹ and 10¹⁰ J/cm;
(b) a microlens array in optical communication with said laser;
(c) a substrate comprising a hydrophobic background layer, and an underlying layer, said substrate being positioned with respect to said microlens array and said laser such that light from said laser focused through said microlens array falls on multiple spatially-separated regions of said substrate;
(d) means for timing a single pulse of light from said laser in operative communication with said laser, whereby said single pulse of light delivers sufficient energy to melt portions of said underlying layer in the regions where said light falls without causing loss of material from said underlying layer, while removing portions of said background layer in the regions where said light falls.

29. An assay device comprising a substrate comprising:
(a) an underlying layer not comprising relief features other than ripples resulting from localized melting; and comprising multiple, spatially-separated functionalized regions thereon; and
(b) a background hydrophobic layer coating said underlying layer between said functionalized regions.

30. The assay device of claim 29 having multiple probe species in each region.
